(19) United States
(12) Patent Application Publication
(10) Pub. No.: US 2025/0244743 A1
(43) Pub. Date: Jul. 31, 2025

(54) Information physics and fractal math for design and practice

(71) Applicant: Gregory Marcus Friedlander, Mobile, AL (US)

(72) Inventor: Gregory Marcus Friedlander, Mobile, AL (US)

(21) Appl. No.: 18/425,251

(22) Filed: Jan. 29, 2024

(51) Int. Cl. G05B 19/4155; G16C 20/10

(52) U.S. Cl. CPC G05B 19/4155; G16C 20/10; G05B 2219/32287

(57) ABSTRACT

The present invention relates to a method for improving processes in various undertakings by employing Key Fractal Elements (KFE). The method involves utilizing KFE to affect change in CT states, a term used to define transitions in dimensional states based on building dimension through compression of fpix. The method can be used to model and interpret matrices of dimensional states defined by iterated equations giving rise to Key Fractal Elements. By applying KFE elements, categorization, prediction, manipulation, and the design of radiation matrices for electronics, solar, thermal, fusion, and radioactive energy applications can be modeled. The invention further encompasses the use of KFE to modify frequency-based systems, enhancing energy generation, transmission, utilization, and storage efficiency. Additionally, base transitions govern matrix composition and interaction, while KFE enables compression, decompression, and dimensional variations in CT states and matrices. The design for reacting and atoms and molecules is optimized based on KFE principles. The controlled absorption and spew of CT state exchanges within matrices, as well as the targeting of AuT plasma, further enhance the effectiveness of the invention.

This patent claims priority from Provisional and Utility patent applications as follows:

| Application Number | Date Filed | Inventor |
|---|---|---|
| 63,441,856 | Jan. 30, 2023 | Gregory Friedlander |
| 63,451,300 | Mar. 10, 2023 | Gregory Friedlander |
| 18/196,930 | May 12, 2023 | Gregory Friedlander |

### TECHNICAL FIELD

The present invention relates to the field of fractal science and information physics in dimensional features from physics to chemistry to more complex structures. The best mode is shown by the determination of where the universe shifts from a fpix based universe to one where it is based on MI based geometry and by changes in base number defining dimensional features including forces, energy, wave, and particle features.

### BACKGROUND OF THE INVENTION

The process involves utilizing the identification of a transition between a curvature based (fpix based) geometry and a MI based geometry at Neutron Bonding, which affects all aspects of fractal structure. Others include the presence of the rule of “3” in proton and neutron fractal structures as well as the presence of the rule of “2” in creating 2\hat{ }n features corresponding to the 3\hat{ }n features and presumable counts beyond according to either the quantum count or MI/fpix features.

It also involves using combinations of the shifting of base numbering systems and the shifting geometries that result.

Major advances in the invention include reconciling empirical chemistry science with the new fractal designs of force, energy, atomic and molecular structure. Empirical technical data in scientific literature is based on highly diverse viewpoints and often inconsistent nomenclature. There are common features in chemistry, but their interpretation and use are often inconsistent if not counterintuitive. One improvement is using the fractal model to limit the number of categories to categorize, modify and utilize empirical knowledge.

KFE can be incorporated into any empirical process, including the equipment to manage that process, to improve the efficiency of energy conversion, the accuracy of signal processing, and the stability of quantum systems. KFE enables increased energy efficiency, improved energy capture and dispersal, and enhanced energy management techniques, enhances reaction kinetics, allows for a way for CT states to be interpreted in a manner that increases reaction selectivity; optimize fission in a manner that improves product yields; allows for the design of molecules and polymer structure formation and improve polymer properties. KFE can be used to interpret or categorize matrices of CT states; for categorization, prediction, manipulation, and designing matrices, including radiation matrices for applications including electronics, solar, thermal, fusion, and radioactive energy use, capture, and dispersal; to modify frequency-based systems, thereby increasing the efficiency of energy generation, transmission, utilization, and storage, while enhancing overall accuracy of results; to leverage base transitions as KFE elements to govern matrix composition and interaction, encompassing spatial, energy, atomic, chemical, electrical, biological, and large structures, applicable across a range of CT states; to efficiently compress or decompress CT states and form matrices of CT states, facilitating desired dimensional variations; to design matrices based on KFE concepts, considering time as CT state dimensional change, energy as CT state change, and the structural aspects of neutron backbones, proton cores, and electron clouds, enabling control over energy release, redirection, and absorption; and design includes the design and manipulation of molecules, including the expansive or contractive features and orientation of atomic or molecular matrices throughout reactions as CT state matrix change occurs; to control the absorption and spew of CT state exchanges within matrices by strategically shaping reaction chamber parts, adjusting injector jets, and exhaust systems; to react chemicals, including fuels, for enhanced matrix changes, maximizing the release of pretime informational change, and optimizing energy utilization and to target AuT plasma type exchanges between electrons and proton oriented positrons, stepped transitions, categorization, chemistry, biology, and other relevant features, thereby improving processes in any undertaking.

### SUMMARY OF THE DISCLOSURE

The invention is a method of working in a matrix of fractal dimensional by generating compressed fractals using fractal manipulation techniques for combining less combined fractals and generating decompressed fractals, further disassociating more combined fractals into less combined fractals. Since the dimensional universe is based on fractals, the earliest and most obvious being based on the iterated equations n=n+1 and fpix, this covers everything. Since earlier patents identified the key fractal elements up to a point, this patent focuses on the new key fractal elements namely the rule of 2's, 3's, the transition at neutron bonding (or the neutron backbone) from fpix to MI dimensions and the resulting processes.

A broad method for analyzing and manipulating different dimensional manifestations of the universe (atomic structural components) is disclosed.

### Key Fractal Elements:

Identifying the fractal information content of a dimensional manifestation using a fractal-based model based on the observed bit fpix as a bit of information for defining geometry and the transition to MI based geometry at neutron bonding using the fractal information content to analyze and manipulate the dimensional manifestation and key fractal elements from the set comprising 2f(n), n=n+1, the rule of 2's manifested as 2\hat{ }n, the rule of 3's manifested in transitions after 3 changes in fractal states, and other transitional rules based n=n+1 or the geometrical fpix and MI, and fpix, Fibonacci (MI) or MI/fpix transitions, where f(n)=fpix, MI, or MI/fpix over 2\hat{ }n compression/decompression sates.

### Shifts in Compression

### Base 2=2\hat{ }n, the Compression Equation

The appearance of circles in the biological form of the overlapping spiral form (reflecting spiral folding or compression) gradually becomes more precise (with greater compression) in alignment with the observed circular form resulting from compressive collapse of systems which gives rise to circles from overlapping spiral forms (f-series spirals) which results from net compressive results.

We can start with the 2\hat{ }n circles, in terms of steps, this is 2\hat{ }4 or 4 such circles. Counting from the overlap outward, the four circles are observed. The first circle secures the overlap of the two F-series spirals, ⅔ of the “1 or unitary first leg.” The second circle contains the resulting neutrons.

Mathematically, this length is 0.333 compared to the unitary first leg and forms the “unitary first neutron length. (UFNL)”

Mathematically, based on the first leg of the Fibonacci series, this derivation is 0.33 (adjacent), 0.67 (hypotenuse); so the UFNL results from a 1:2 ratio within 2\hat{ }n circles where n moves from 1 to 2 which corresponds to a right triangle with “the square” of the third, opposite leg equal to the length of the squared hypotenuse less the squared adjacent leg. Put another way, the square of the opposite leg is equal to the overlap/2, and this is the radius for the neutron.

0.33 is also 1/fpix for n=2. This diameter is also the overlap and ⅔ of the length of the leg. This is fpix for 1+1/fpix(2) which is the first solution for this equation used for calculating pi. Accepting the 1:3 diameter of the proton to neutron, the first leg of the f-series is ⅓ the diameter of the proton, and ⅔ of the first leg is the diameter of the neutron, a fraction of the proton fpix overlap which is not observed directly.

The length of the MI first leg then relates to the Proton curvature based equivalent according to this: ⅓ of the diameter of the proton=diameter of the neutron.

The offset of compression for neutrons is ⅔:⅓ (the overlap) equal to the diameter of the neutron. This 2/9^{th} also corresponds to the 2×9 bundles making up a base 9 proton.

The theoretical transition, supported by the ratio of gravitational force relative to the strong force, suggests that ct1 to ct3 are the 3 steps of compression which suggests that at the neutron, there is a shift to a dual compression, based on continued fpix compression of MI based mathematics which then carries on to compression. These shifts and the information exchanges can be targeted to get useful results.

### Rule of 3's in Material, Including Crystal and Explosives

The rule of 3 originates from the same place as base 2, which counts at 2\hat{ }n which is also preserved, namely (x=n+1 for n=2 for base 3 and n=1 for base 2).

The intersection with the squared Energy transition can be captured and controlled with 2\hat{ }n and with 1:1.3 matrix transitions. These form pockets within the transition due to the math that controls them. These are visible in certain explosions, snowflakes and flowers as the interplay between fpix and MI transitions define structures.

While it does appear again (2*3=6) in atomic modeling, this is more specifically related to the transition to MI modeling at neutron bonding.

These transitions can be targeted for any reaction where such explosive or crystalline transitions occur shows other fractal targeting in blue where ⅓ in from the outer shell identifies an area of concentration and 2× this ⅓ indicates another key area. This reflects a process for modeling the compression information for fusion or for explosive or armor design.

Shifts in f(x)

Two visible shifts are present at the atomic level. 1,3,5, possibly 7 for protons, then back to 5 for the proton to neutron transition and 2, 6, 10 the shifting to base 6 (6-unit modules) for neutrons. Like the 7-5, the neutron counts of multiples of 6 works both as an 18-unit backbone and a backbone comprised of 3 sets of 6.

The next shift at 24 also corroborates with either an MI calculated backbone or as 4 sets of 6, but past that the count works with sets of 5.5 (acting either as 5.5 units or 6 presumably). This also reflects the rule of 3 changes followed by a transition.

This relates the three features of information (fpix), f-series overlap and exponential compression. One can imagine this as the spirals (f-series) collapsing within exponentially compressive areas.

When we look at hydrogen to neutron fusion, we can see it as 2\hat{ }n compression in this fashion:

Two neutrons of ⅔ of ⅓ proton length (UFNL) fit within a circle double the diameter (2\hat{ }n sized) the UFNL. Assuming a base 9 proton geometry (base 7 and geometries seem less likely), the 2 base 9 arms of the neutron are unexploded reverse exponentially into a neutron under this scenario. The electron area is incorporated into the resulting neutron and the resulting base numbering of 10 is from maintaining the value of n=4 as it changes from 1, 3, 5, 7, 9 (−3, −7) to 1, 2, 3, 5, 8.

The 3:5 ratio can describe area or features, but it also defines the number of protons necessary to balance a larger number of neutrons on compressed sets of 6.

Controlling change based on compression and decompression of information and changing the matrices in which that is happening as opposed to other features; particularly in this case in targeting places in a matrix for changing folding vs unfolding and the absorption of information to change the way that the information transitions occur.

Fulcrums, about which compressive folding of information states are balanced, are also KFE which can be targeted.

Identification of energy and force as transitions between compression based on the broad equation x=2f(n)\hat{ }(2\hat{ }n) where changes in x, e.g. from n=1 to n=2 are observed as force and particularly where these transitions change at neutron bonding between f(n)=fpix(n) to f(n)=MI(n). The steps between 2\hat{ }n compression and irregularities allow forces to be varied.

This includes fractally relevant shapes such as shifting base numbering, six or 14-sided structures representing atomic inflection points and pre-atomic, proton inflection points and repeated patterns such as those shown in the proton and neutron counts. This includes targeting of fractally relevant patterns, such as the six sided pattern of two dimensional protons around a carbon neutron core and the natural breaks within atoms giving rise to the number of bonds possible and the strength of the bonds based on the neutron backbone and surrounding fractally relevant areas of protons and electrons and A-Positrons sharing information between the electrons and the protons along with similar sharing effects and their transitions within the matrices of gradually changing compressive and dimensionally relevant states.

One KFE is targeting dimensional change in place of time at the level of photons, although the transition is gradual and includes all dimensional change below the level where it manifests. This includes a loss of dimension or a building of dimension from ct1 to ct5 taking quantum steps at full ct states including the shift in dimension building to that based on MI at neutron bonding. Replacing time with net compressive and decompressive changes allows better modeling and using this within matrices allows for better definitions of relativistic effects as shown with relativity itself being replaced with pretime change within a matrix lowered as the pre-time changes are stripped by translation into movement within a larger matrix or being pulled off by gravitational folding.

In addition to Fibonacci (1, 2, 3, 5, 8, 13) and fpix (1, 3, 5, 7, 9), other KFE includes:
1. the building sum (1, 2, 5, 8, 3, 2, 7, 12) results from the absolute value of “fused” values of fpix.
2. The sum of the digits function is considered unrelated to the MI/Fibonacci series when using a quantum count. The importance of the sum of the digits originates with the quantum count, n=n+1 which is the power behind the model. This is an exact fractal relationship of the simplest kind. Between the quantum count and MI lies fpix. Fpix results can include: 3 (not expected), −1, 1, −3, 5, −7, 9, −11, 13 etc. The relevant solution, if we look at the sum of the digits for two places is 2, −2, 2, −2, etc. For three places, ignoring the 3 in the series, you get −3, 3, −5, 7, −9, 11 or the inverse of fpix.

Better modeling of the resultant magnetic matrix changing in a pre-time quantum steps to give force and in combination with key fractal elements or combos of key fractal elements to form energy as “pre-time” quantum change hidden behind the “wall” of time, thus arising as a form of “stop-frame animation” due to changes in compression at the photon level of compressed states. Because the universe is based on f(x)=fpix up to neutron bonding and f(x)=MI beginning with neutron bonding and because we have the compression equation arising from binary initial compression, we can use the equation 2f(n)\hat{ }(2\hat{ }n) to compare gravity where f(n) changes from for fpix changing from n=1 (1) to n=2 (−3); to the strong force where f(n)=MI for n=4 (1, 2, 3, 5, so 5). The numerator of pi reflects the building of dimension. Calculating the result using fpix for one gives 1-dimensional curvature, 2, 2-dimensional curvature, etc., since at the neutron there are only 3 dimensions the single suggested result is 1, 2, −4 from the equation (2x(−1)\hat{ }(x−1). That would make the four-dimensional architecture of a black hole defined by a numerator of 6. Another suggested model for the numerator is −1, 3, −5 but the AuT model indicates that dimension shifts at the neutron, so perhaps it shifts from 3 to 4 or from −5 to 4 or even from 7 to 4.

Everything being fractal, the process can involve categorizing, tracking, predicting, and targeting changes in fractal features of the matrix for energy manipulation, material manipulation, energy storage, energy generation, machine learning, chemistry, predictive science, or quantum computing.

This involves a. identifying the compression states within a data set; b. utilizing key fractal elements within the identified compression states, and; c. tracking the fused compression states for things from data management in computing functions to chemical transitions.

In one embodiment, the computing functions include sorting, organizing, and tracking data in search engines for key fractal elements within the data. At the smallest level measurement based on fractal states allows measurements below the level experienced as space. Another extreme is treating energy as a transition between pre-time fractal compression states and post-time fractal compression states.

Treating time as the result of quantum dimensional change in fractal states within the matrix where the fractals transition over quantum dimensional changes leading to time, allowing relativity and probability to be treated as the net pretime change within one matrix relative to another. In this way a person traveling faster with a large matrix, sacrifices internal pre-time change for change within the large matrix. Similarly, gravity results from folding pre-time information thereby taking away the pretime change which is viewed as time within the individual.

Because we now have different dimensional transition baselines for f(n) at atomic (neutron bonding) and subatomic level we can modify the interactions of the two different dimensional states and within the two dimensional states to create desired transitions in fractal compression processes to change transitional and net features of the different matrices of compression states between lower and higher compression states from the group comprising increasing compression, decreasing compression, destabilizing compression or stabilizing compression.

One of the embodiments further comprises encouraging fusion by facilitating the transition from non-bound to bound neutrons by focusing the staggering of neutron concentrations internal to proton concentrations to mimic fractal compression of dimensional features. Employing compression and decompression based on changing the arrangement of fusion steps and scales based on key fractal elements.

Treating neutron bonding based on neutron fractal elements balanced on either side by lower compression states, most notably Protons, it includes lower information states as fulcrums balanced by the two arms of Neutrons and now it includes transitions in geometry.

The neutron and proton concentrations are separated based on a Fibonacci ratio between the two on either side of the fulcrum, now this is no longer an MI ratio, but is instead an MI to Fpix ratio. In addition, other results are the presence of shifting base numbering within the transitions of either fpix or MI.

The process of designing fractal changes further comprises designing fractal changes to enhance a process from the group consisting of energy manipulation, material manipulation, energy storage, energy generation, machine learning, chemistry, predictive science, and quantum computing; to enhance a process from the group consisting of energy manipulation, material manipulation, energy storage, energy generation, machine learning, chemistry, predictive science, and quantum computing, compressing, decompressing, increasing ct states within the matrix (as by combining two matrices), changing the net fuse length of the matrix, changing the absorption of the matrix, changing the spew of the matrix and identifying a ct state as an identified ct state within the matrix and changing the ct states making up the identified ct state.

Manipulation includes changing the rate of lower compression state modification of key fractal elements within the matrix. It includes targeting the pretime change features of energy and the post time features of atomic structures to maximize the absorption or reflection of features embodying pretime change along these different dimensional pathways. To increase compression, you take combination of states which are tending towards compression, and you put them together with other similarly situated and inclined compression states. Decompression occurs the same way.

The key fractal elements (KFE) are targeted in changing the ratio of changeable fractal states to non-changeable fractal states at different points within the matrix for changing the rate of lower compression state exchanges between higher compression states, targeting fractal changes to enhance a process.

Ct state is a way of distinguishing fractal changes and in this case ct5 and transitional (neutron bonding) states are those which have MI bonding. A list of how these can be used includes to following:
(1) to model methods to affect change in CT states and interpret or categorize matrices of CT states.
(2) to utilize for categorization, prediction, manipulation, and designing radiation matrices for various applications including electronics, solar, thermal, fusion, and radioactive energy use, capture, and dispersal.
(3) to modify frequency-based systems, thereby increasing the efficiency of energy generation, transmission, utilization, and storage, while enhancing the overall accuracy of results.
(4) to leveraging base transitions as KFE elements to govern matrix composition and interaction encompassing spatial, energy, atomic, chemical, electrical, biological, and large structures, applicable across a range of CT states.
(5) to utilize KFE to compress or decompress CT states and form matrices of CT states, facilitating desired dimensional variations.
(6) to design matrices based on KFE concepts, considering time as CT state dimensional change, energy as CT state change, and the structural aspects of neutron backbones, proton cores, and electron clouds, enabling control over energy release, redirection, and absorption; such as designing, based on KFE, the proximity of states and the nature of the intervening matrices between reactant matrices, rate of injection and alignment by shaping of the reaction chamber parts (cylinder, piston, injectors); the number, location, and series of injector jets and exhaust and by controlling the mixtures of different CT states used to maximize desired matrix results, for example to maximize the heat energy going to expansion, contraction or other desired post information exchange matrix.
(7) to incorporate KFE to facilitate the design and manipulation of molecules, including the expansive or contractive features and orientation of atomic or molecular matrices throughout reactions as CT state matrix change occurs;
(8) to control the absorption and spew of CT state exchanges within matrices by strategically shaping reaction chamber parts, adjusting injector jets, and exhaust systems
(9) to react chemicals, including fuels, for enhanced matrix changes, maximizing the release of pretime informational change, and optimizing energy utilization as pretime change at various levels of information compression and creating alternating balancing to stop reactions and unbalancing to provide catalytic and other reaction mechanism.
(10) to employ KFE to target AuT plasma, stepped transitions, categorization, chemistry, biology, and other relevant features, enabling advancements in various fields
(11) to leverage KFE to achieve balance within matrices, utilizing fulcrums, absorption, and spew mechanisms, and leveraging fractal alignment patterns for efficient CT state interactions
(12) to utilize KFE to design, control, and optimize the proximity of states, the nature of intervening matrices, and the mixing of CT states for desired matrix results.
Manipulating pretime transitions through the application of KFE offers the potential for more efficient fusion and fission processes, enabling the development of sustainable and clean energy sources. Moreover, the incorporation of pretime principles in electronics, batteries, quantum computing, and AI can revolutionize these fields, leading to breakthroughs in information processing, energy storage, and intelligent decision-making systems.
(14) fractal design of matrices using time as CT state dimensional change, energy as CT state change, the neutron backbone, the structure of resulting MI based neutron backbones, fpix based proton cores, and electrons as particles in pre-time positions manifested as energy as pathways to chemistry, to increase and control the release of energy, redirection or absorption of the energy.

### Fractal Modelling

Fractal modeling using KFE is the core conceptual inventive framework.

The values of fpix as n changes from 1 to 5 are 1, −3, 5, −7, 9 derived from the fractal formula:

$$fpi ( x ) = [ ( - 1 ) \hat{} x ] + 2 x * [ ( - 1 ) \hat{} ( x - 1 ) ] .$$

When variable 2f(n) is used for f(n)=fpix, the proton counts, and full electron orbitals and semiconductors can be derived. Fractal math here shifts from fpix to multiples of 9 after the first 3 full atoms (from 2×9 to 3×9, 4×9, and 5×9) after the first three even-noble gas solutions (interspersed with the odd-“semi-conductor” counts) in keeping with the fractally relevant rule of three in n=n+1 modeling.

The fewer protons at radon are believed to be based on the lack of room around the neutron backbone. The neutron count shows the transition from a universe governed by fpix (curvature) to a universe governed by MI (Fibonacci).

The fractal origin of proton and electron counts is displayed based on 2f(x) where f(x)=fpix reflect the balancing (the factor of 2) critical to the structures defined; 2*1=2, 2*−3=−6, 2*5=10, etc. The “even” results give rise to the noble gases, and the “odd” results define the semi-conductors.

Proton Count and Electron Orbitals reflecting FPix.

| fpix | orbital size | Element |
|---|---|---|
| 1 | 2 | He |
| 3 | 6 | C |
| 5 | 10 | Ne |
| 7 | 14 | Si |
| 9 | 18 | Ar |
| 18 | 36 | Kr (2 × 9) |

This is reflected in proton and electron counts, so the base numbering and therefore the dimensional state for electrons and protons is different from neutrons where the completion of compression changes the dimensions from 2 to 3 and the base numbering is completed to that changing the dimensional structure in which protons are held with electrons encourages them to collapse into the neutron form in the same way that ct5 states are formed from completed ct4 (neutron) building to create a 4 dimensional framework with the associated non-MI, non-fpix compression, but a hybrid like the MI is a hybrid defined by the circular areas defined by fpix in the form of pi as the geometry transitions from pre-circular to circular which can be copied to encourage fusion of neutrons.

Neutron count based on application of overlapping MI spirals and measuring the length of the arms:

| | | | length | Running | | |
|---|---|---|---|---|---|---|
| | | proportional | for 2 | Total | | |
| Compression | segment | correction | arms | N | observed | |
| new | length | 0.33 | x2 | calculation | neutrons | Element |
| s1 | 0.33 | 1 | 2 | 2 | 2 | He |
| s2 | 0.57735 | 1.749546 | 3.499093 | 5.499093 | 6 | C |
| s3 | 0.713644 | 2.162558 | 4.325116 | 9.824209 | 10 | Ne |
| s4 | 0.709006 | 2.148502 | 4.297003 | 14.12121 | 14 | Si |
| s5 | 1.430501 | 4.334851 | 8.669702 | 22.79091 | 22 | Ar |
| s4 + s5 | 2.139506 | 6.483353 | | 22.79091 | 48 | Kr |

### DESCRIPTION OF FIGURES

FIG. 1 shows two different arrangements of neutrons within a fractally relevant Helium atom.

FIG. 2 shows one of the two embodiments of FIG. 1 with electro orbital areas identified.

FIG. 3 shows the resulting structure of a carbon atom.

FIG. 4 shows the resulting structure of a neon atom.

FIG. 5 shows one version of the resulting structure of an Argon atom.

FIG. 6 shows the proton fractal model.

FIG. 7 shows the neutron fractal model for larger atoms.

FIG. 7a shows an alternate structure for an argon atom.

FIG. 8 shows how fusion can result from fractal targeting at the proton to neutron level.

FIG. 9 shows how information is released in a carbon and oxygen reaction.

FIG. 10 shows how information is released in a hydrogen and oxygen reaction.

FIG. 11 shows how fractal modeling can be targeted in a reaction.

FIG. 12 shows how 2\hat{ }n and rule of 3 changes appear in compression changes.

FIG. 13 shows the process steps of how this invention is used.

### DETAILED DESCRIPTION OF THE PREFERRED EMBODIMENT

Traditional figures for patents can be eschewed in favor of math steps expressing the universe as fractal representations of iterated equations. The universe is a process and to show the process as fixed elements is to ignore the underlying mathematics.

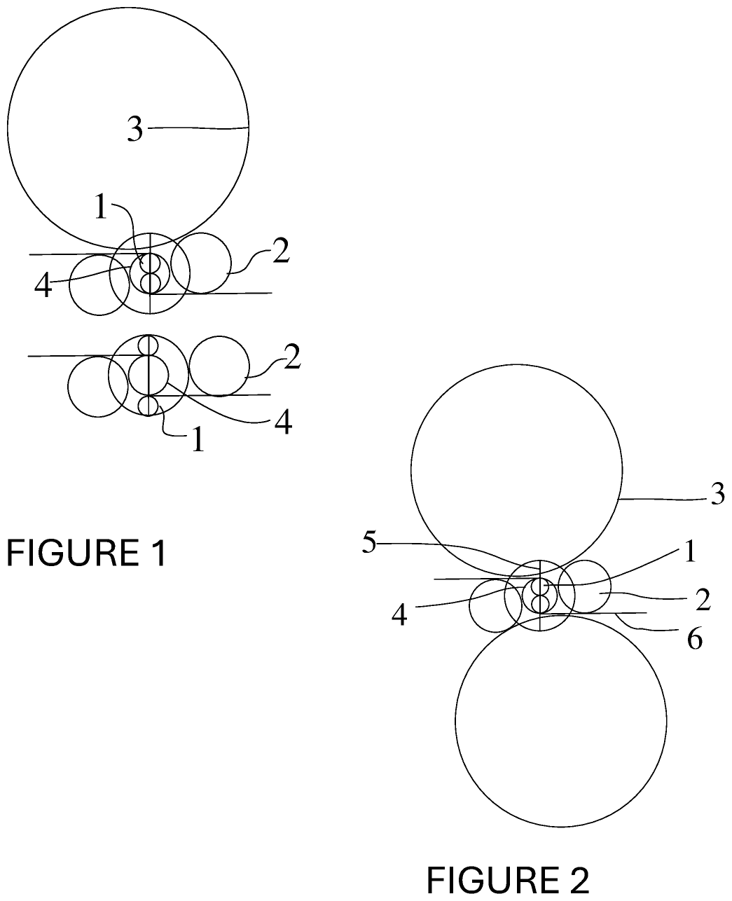

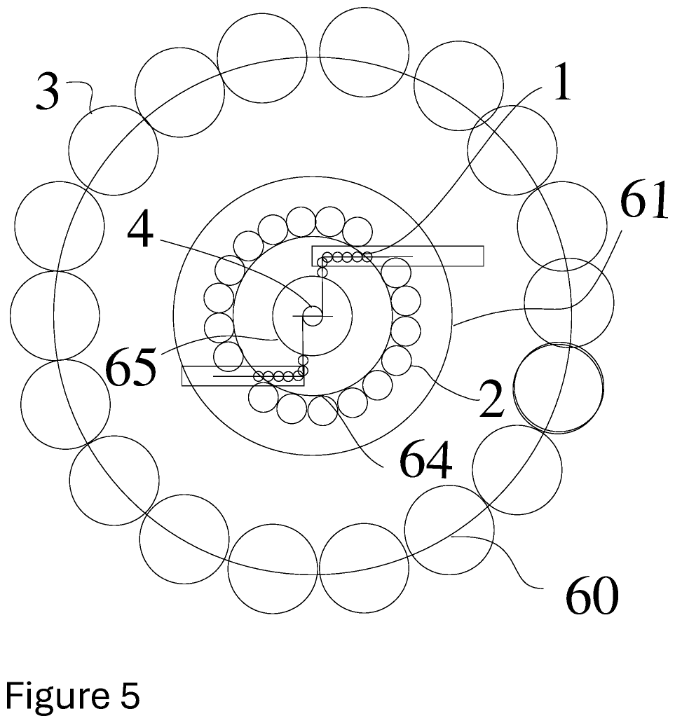

FIG. 1 through FIG. 5 shows the fractal effective size of neutron backbone composed of neutrons 4 which are viewed for purposes of fractal consistency of relative numbering as ⅓ the size of the protons, a relationship reflected in force exchanges as well as atomic design. Also shown is the transition to MI geometry as the overlapping MI spirals which form the backbone are distinguished from the circular rings of protons 2 and electrons 3. While the sizes of the neutrons and protons are fractally relevant, if not precise, this is not the case for electrons which are relatively small, but which occupy larger areas over time, so this occupied area is shown to give an example of how these small units might balance the protons 2. Modeling suggests that positrons defined as one half of electron pairs extend from the protons 2 and are responsible for their charge and occupy part of the area of the electron 3 and are not separately identified since they remain largely a theoretical fractal element.

Using this modeling, two different neutron cores, the inner ending with a 10-unit backbone at the circle representing 2\hat{ }n for n=3 with the neutrons being M the radius used for the n=1 circle all of these being based on a 2\hat{ }n radius. The extended backbone (22) ends at the circle representing 2\hat{ }n for n=4. Two different proton cores are shown, the inner core of 10 protons for Neon (n=3) and the outer core of 18 protons for Argon. Exterior to this is the area in which electrons can be found, the number of electrons corresponding to the number of protons as in the model individual electrons balance the protons in ground states. The size of the electrons is relatively minor compared to the size of the protons, but they occupy multiple areas over time giving them their cloud-like appearance.

FIG. 1 shows a representative view of one form of ct1 solution folding and FIG. 2 shows a representative view of ct3 to ct4 compression. Further, FIG. 1 and FIG. 2 are in detail explained with the following process:

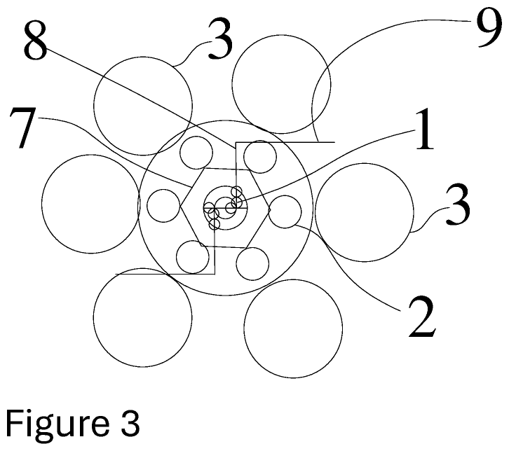

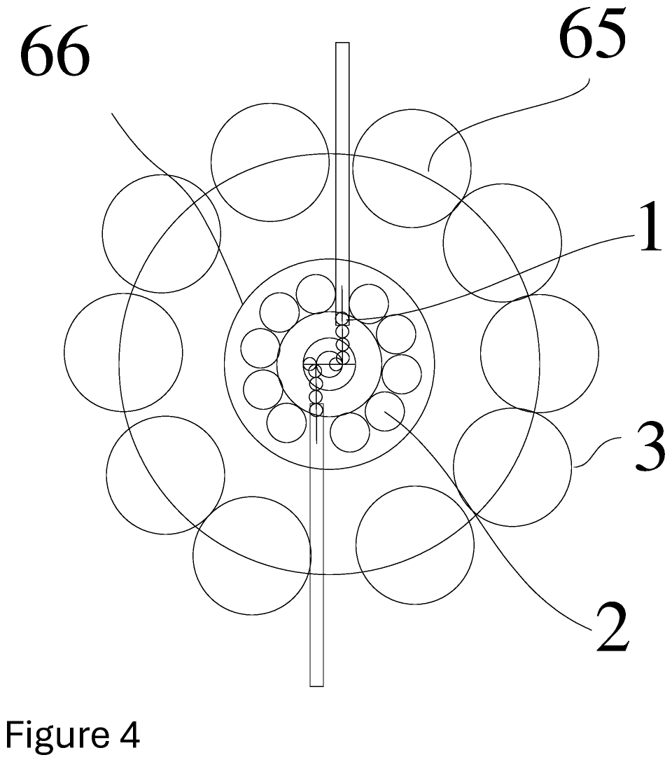

FIG. 2 shows possible geometries for Helium. Note that the neutrons 1 can be inside the first circle 4 or on either side of the first circle at the extension 5 from the area of overlap shown within the first circle 4 so that the length of the spirals within the first circle 4 and that in either extension 5 form the first legs of the MI spirals. The resulting geometry shown in this figure explains the staged compression targeted for fusion. Protons 2 can be outside the first circle 4 or outside the second circle 5 given the geometries which are possible with just two neutrons. Perhaps a hint can be found when electrons are added and close in the area around the 2-unit neutron shown in FIG. 3. Here The second leg 6 of the overlapping MI spirals 6 can form the fractal base for the two electrons 3 which balance the atom.

The electron size is a small fraction of the proton size, and both protons and electrons are largely 2 dimensional in layout since 3 dimensional folding is more pronounced with the neutron, so the actual size of the electron can be wound smaller, but takes up more room, presumably, the more unwound it is.

This clearly shows the collapse as the Proton-positrons giving the larger area collapses into the neutron core and the geometry and changing base numbering associated with the neutron collapsed geometry makes more room for the outliers to be within the smaller area.

The six-sided hexagon 7 reflects forces equivalent to the Pauli exclusion principle, but in this case is likely the result of common spew of information balanced by common absorption of information of a different fractal compression.

The drawing shows the backbone for Neon. The spacing, due to the overlap, is such that the neutron is ⅓ the size of any one arm. So, either two may be in the central area, or two may be on either ⅓ of the line extending from the central circle. The 0.33 length of the neutron (⅓ the arm length) is the unit used to measure the other lengths of the backbone. To additional lengths of the second arm are important, the first within the second circle and the third inside of the third circle defining carbon and the base for larger atomic structures and Neon respectively as can be determined by counting the neutrons. The number of protons that can fit around the third circle and still leave space for two-dimensional extension of the MI spirals is shown. The PTE is defined by the number of neutrons, but since stability is possible with other neutron counts with different proton counts within certain ranges, although unstable, and since the size of the proton shell plays a large role in bonding as discussed below, the traditional classification has validity. Carbon 16 would have the same backbone as Neon. For this reason, it decays into a larger atom, typically Nitrogen and in some cases from Nitrogen to Oxygen (taking 2 sets of the 4 extra neutrons) and Carbon (6 neutrons) so that it is actually gaining protons or neutrons have to break down into protons to get stable atoms.

You can leave this core of neutrons in place and extend it to the edge of the 4^{th} circle as shown in the next figure in which case the protons are the protons that can fit into the 5^{th} circle. As with Carbon, the Semi-conductor is defined by the place where the arm containing the neon neutrons is filled with neutrons. In such a case, the Protons are those which can fit outside of the intermediary circle defined by the diameter between the size “2” MI leg.

The base numbering transition is important to fusion of protons.

Forcing, for example, a base 5 geometry on base 7 protons through shaping virtual chamber collisions.

Information is shared at an overlap, exchange of information. This reduces the total amount of information at least at the center by ⅔ divided by 2 or ⅓ since each still has half of the ⅔ overlap at MI.

Ordering components or creating areas where the components can be ordered. This might require a compression-oriented core of neutrons with added compressive features interior to an outward shell to allow Protons to collapse into Neutrons with the required Proton balancing and orientation means to encourage F-series alignment of the newly formed neutrons.

### Proton Count Fractals

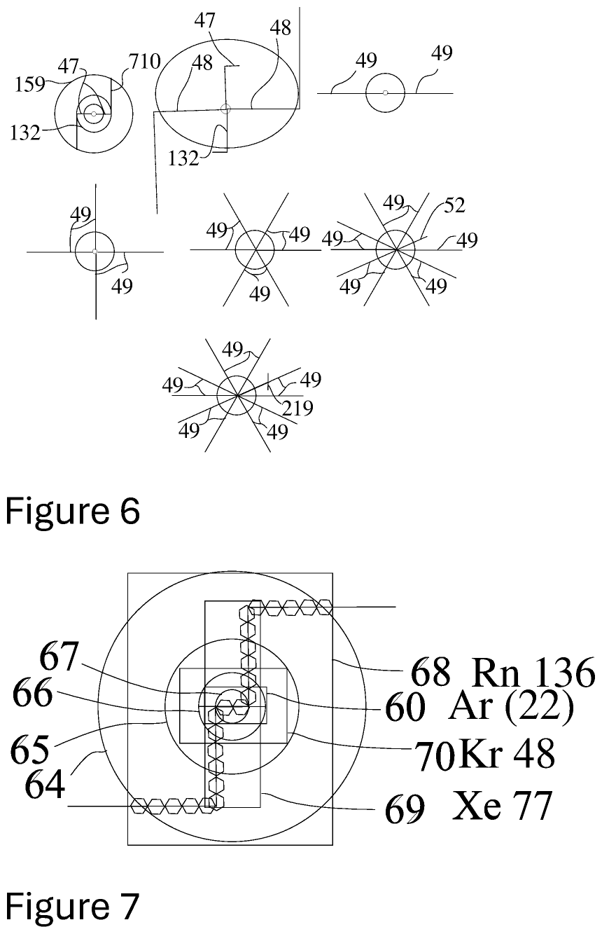

FIG. 6 shows Proton count according to the model, showing the geometry follows fpix for both electrons and protons.

FIG. 6 displays two balancing hydrogens 47 for a helium atom, 132 is identified by the effective radius of the proton core, the three negative value arms 710 with two of these waiting to be filled to give carbon identified by the potential carbon core radius 159. Neon has five balancing arms 48 and Argon where the scale has been changed has negative value arms −7 would yield the next semiconductor, Silicone. For Argon there are two balanced arms 49 giving the 18 protons observed. After Argon, there are 4-9-unit arms 49 for Krypton, 6-9-unit arms 49 for Xenon, and for Radon there are 9, 9-unit arms plus a broken arm which is 5-unit unbalanced arm 52. While shown this way for clarity, balance suggests that the broken arm and one of the 9-unit arms 49 would each be 7 units and balanced, although different from the 9-unit arms. Each of these 7-unit arms (not shown) might come off one of the 9-unit arms, by way of example. The repeating “9's” indicates that the repeating feature of the neutron backbone is that shown for Argon (2×11=22) as opposed to the balanced “5's” of Neon although radon indicates it might be the balanced 5's with a bridge of 2×1=2 between the two neutron backbones. A larger proton balancing in Uranium is displayed with 10 9-unit arms 49 and at the place where the unbalanced arm 52 would have ended there is a helium proton pair 219. The relevance of this modeling will be discussed in more detail. The broken Radon may correspond fractally to the electron-proton interface.

### Neutron Modeling Around MI

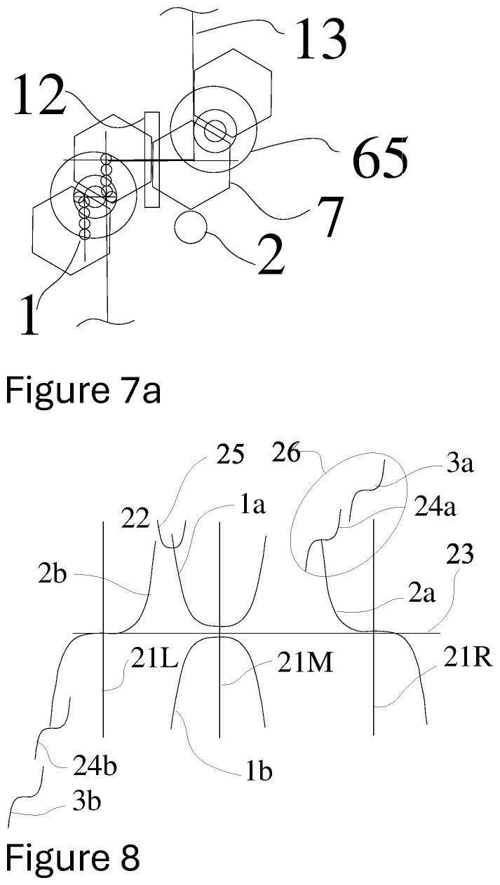

FIG. 7 shows neutron modeling around MI for larger atoms. Argon works based on overlapping spiral calculations at 22.7 calculated.

Design can be around reconciling dimensional modeling as shown in FIG. 7a where design is arranged about a truncated set of overlapping spirals, it is also possible that argon can achieve a six-by-six geometry by sharing two neutrons in the common face 12 of two six-sided neutron areas 7 at overlap 7. This concept of overlap could provide an alternative basis for the Xe and Rn counts since these are fractal relationships. While a single proton 2 and neutron 1 are numbered, all of the common parts are not shown to prevent the drawing from losing clarity.

FIG. 7a which shows the interaction between three dimensional neutrons based on MI geometry and two dimensional fpix based electrons and protons.

Fractal transition from individual neutrons to blocks of 6. In larger atoms, the Carbon core 5.5 to 6 variation can be used to design the atoms as hexagonal blocks (of 5.5 to 6 neutrons) of neutrons arranged in unitary blocks within the expanded model from individual neutrons (up to Argon (22 Neutrons, 18 Protons) to units of 6 from Argon to Radon. The fractal size reflects the space taken up in MI modeling of the sets of 6. The areas of overlap or area of association are the same as with individual neutrons proportionately.

In this case, in sets of 6, Argon, in box one 60 is 4 sets of 6, Krypton in box 70 is 6 sets of six.

Xenon is the first special, more theoretical case since it is a balanced “14 units” but these appear as all 5.5 units as opposed to 6 units (14*5.5=77) in box 69.

Radon follows the Xenon pattern appearing as 24 balanced units which appears to be 16 units of 5.5 (88) with 8 units of 6 (48N) shown in box 68.

Observation is that that this second shift in MI compression occurs at Argon which works in either form of MI modeling. In this case, for example, item 4. Is replaced with item 67 but fractally, these represent 2\hat{ }n compression changes, which is the area in item 66 is 2\hat{ }n times that of item 67 and the radius of item 66 is twice that of item 67. So we have an inner circle 67 which is of sufficient diameter to hold ⅔ of the first legs of two MI spirals, fourth circle 66 with a diameter twice that of inner circle 66, third circle 65 with a diameter twice that of fourth circle 66, and second circle 64 which is twice the diameter of third circle 65.

Isotopes can change the overall structure; this modeling is to show the most stable form of the atoms.

The problem that arises with the shifting base numbering is that “n” under fpix is 1, 3, 5, 7; so, 7. This would mean if we're using n=7 up to neutron pairing and then moving to n=5 (1, 2, 3, 5) which reflects several changes.

FIG. 8 shows how fusion can be modeled in proton to neutron compression. 1a and 1b are neutrons formed by protons 2a and 2b being combined along reaction line 23 at middle perpendicular point 21M which is between left perpendicular point 21L and right perpendicular point 21R. Two different dimensional compression changes, one for the electrons 24b and 24a which combine to make a collapsed electron 25 in the neutron or with positron 24a and 24b, the two protons being transitioned to two neutrons in this drawing by taking advantage of the shifts in geometry of the two halves, those halves on either side of reaction line 23. Whether these transitions can be accomplished through the combination of the items in 26 to get to 25 or getting 2a and 2b together to form 1a or 1b depends on the method used so these are shown separately.

The scale of the different transitions is based on the transitions required and the alignment transitions required to abide by the rules of the mathematics of the iterated equations of the key fractal elements.

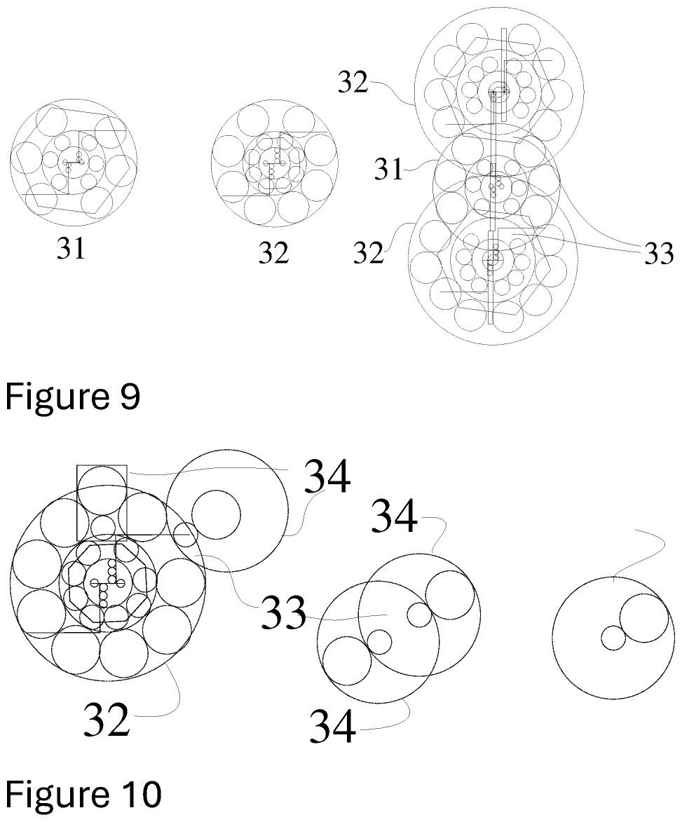

FIG. 9 and FIG. 10 show Reactions are shown to scales, but as shown by fpix and MI modeling of the protons and neutrons respectively, you have to balance the entities based on the reaction elements which can naturally come together and react if a great stability is yielded and the parts are allowed to rearrange to the more stable form. Areas where information is released 33 when carbon 31 and oxygen 32 are combined show where with isotopes the carbon features remain predominant (although less stable or balanced) since the bonding at pre-fusion levels occurs at protons. The heat allows the transition of evenly distributed protons to shift to compressed protons providing space for bonding and this can be copied for fusion at earlier compression levels, the neutron backbones need to be aligned and this is necessary, and presumably easier, by following similar alignment for electrons and proton based positrons (electron units extending from protons to share information with electrons).

Ways to do this are shown with various dimensional change arranged to push the elements together in the order and scales required by transitions in dimension and base numbering required by the iterated equations defining the key fractal elements.

This release of information can be modeled with all the elements of a matrix, the reactants, catalysts, pretime change (energies), information exchanges (forces), geometries and the containers. Where hydrogen is a part of the reaction, such as with hydrogen fuel cells, the exchanges and dimensional changes are particularly relevant.

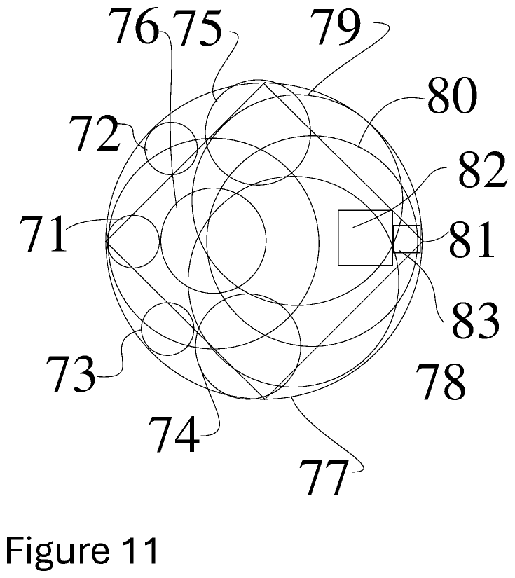

Pumping at kfe based scale and direction is shown in FIG. 11. While shown for fusion, the pathway design for force transitions and chemistry including catalysts are modeled using the same methods. In this case changing compression steps move into a corner 81.

Along with a real chamber 14, a “virtual” chamber is defined by a series of magnetic, photonic or explosive pushes beginning with middle first push 71, centered with top first push 72 and bottom first push 73 exponentially increasing with middle second push 76 centered with top second push 75 and bottom second push 76 and third middle push 80 balanced by third top posh 79 and third bottom push 78 into a compression notch 81 where the first reactant 82 is exponentially compressed around second reactant 81, for fusion 81 would be neutron heavy and 82 would be proton heavy, heavy referring to a greater number than would otherwise be balanced according to KFE modeling.

The arrangement of the pushes and their impacts would be designed around transitions desired for various KFE.

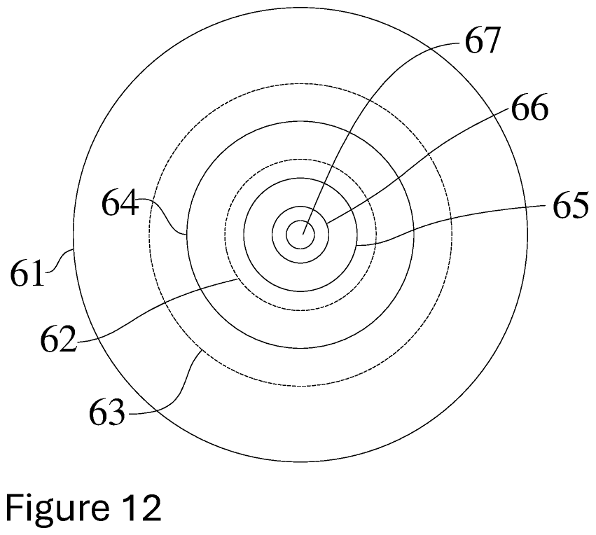

FIG. 12 shows 2\hat{ }n changes with intermediary steps. Compression does not involve vacuum, but the transition of information from non-compressive to compressive in an area.

2\hat{ }n compression and the rule of 3 can be seen in looking for changing information density after a fusion explosion or neutron star level explosion in a vacuum. Modeling shows that in the absence of fractal information transitions, the outer circle 61, beyond which information concentrations are too dispersed in significantly more random appearing states, you can establish a base. Moving inward, you see the 2\hat{ }n compression states, second circle 64, third circle 65, fourth circle 66 and inner circle 67 which appears as solid energy. There are intermediary transitions, most obviously between one third max 62 which is ⅓ of outer circle 61. If you take item 62 and square it (multiply it by itself) you get third max squared 63.

Between items 62 and 63, it is seen as devoid of visible information which is concentrated between items 63 and 61 or between the outer circle 61 and ⅔rds of item 61.

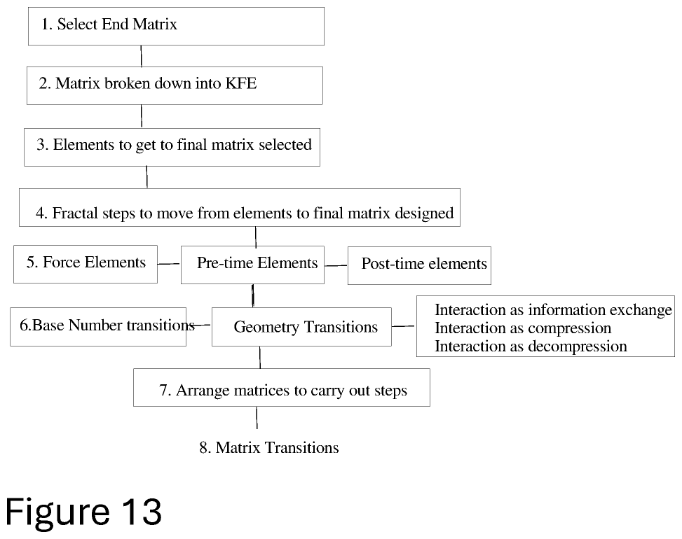

FIG. 13 shows the process used in the preferred embodiment.

In broad terms the invention is described as using information physics, in this case using fpix as the state of information and fractal mathematics, in this case fpix being the iterated equation primarily responsible for designing transitions in order to design and carry out various processes.

This is a method for analyzing and manipulating information states defined as parts dimensional features comprised of combined solutions of fpix to design electromechanical, physics and chemical interactions in structural components.

It can start by categorizing any information as fractal elements including force and matter. To use this to sort and analyze information is one way of beginning the reconciliation of pre-fractal design and post-fractal design according to this method. For the purposes of FIG. 13, we are going to look at carrying out a process for a specific result. In this case the “final result” is defined as an end matrix of information states in a particular arrangement so step 1 is to Select the End matrix. This could be two protons fused to form two neutrons, by way of example, but the surrounding containment vessel and atmosphere would be part of the end matrix, so the second step is to break down the end matrix into Key Fractal Elements. The third step is to determine the Elements to get to the final matrix selected. This is then broken down into the fourth step which is to define all of the fractal steps to move from the elements to the final matrix designed. Both the Third and Fourth steps can include looking at those elements viewed as broken down in the first step as separating force (the exchange of information states), those occurring pre-time and giving rise to time, and those post time elements.

To accomplish this effectively it is also required that the third and fourth steps take into account base number transitions, geometry transitions (particularly fpix to MI based geometry, but also intermediary steps in the transition) and interactions related to information exchange, compression and decompression of information states as shown in the sixth step.

To carry out the process, if it goes beyond design, matrices are arranged in step 7 to carry out the steps according to the method taught and the matrix transitions are carried out in step 8.

Identifying the KFE fractal information content of atomic dimensional manifestations using a fractal-based model, on the observed bit fpix up to neutrons; Identifying the KFE fractal information content of atomic dimensional manifestations using a fractal-based model, on the observed bit MI at neutron bonding; and applying the KFE fractal information content to analyze and manipulate any dimensional element in the universe can be carried out according to this broad process.

### Specific Examples Include

Fusion: Fusion requires conditions of compression with balanced alignment so that when Neutrons form, they are balanced and supported by protons along fractal lines. Neutrons can be formed with 2\hat{ }n compression steps using a means from the prior art to generate free neutrons put into a central area and compressed with protons to balance the fractal structure according to the MI to Fpix ratio.

These atomic scales are huge if you use uranium 235 as a source of neutrons, many are generated even with a small amount and with limited decompression, but the distances are very small and hence you need magnetic fields to guide the neutron in place and lasers to concentrate the protons around them to give them stability along with their paired electrons.

At atomic levels, only pretime elements, such as magnetic compression with magnets aligned to create compressive forces and to create forces to provide pressure exterior to push elements together work at scales not possible with larger ct states to provide information channels of as small ct states as possible to take advantage of fractal compression alignment. Compression using photonic lasers can be used to the same effect, using the concepts of KFE to create fractal environments where the combinations of ct states are used for balancing and compression or decompression along fractal lines.

This involves staging along fractally mandated steps. The process includes a concentration of fusion elements in the order set forth utilizing equipment to sequentially generate the elements and, in the ratios, determined with a capsule for the concentration of the fusion elements according to the order set forth utilizing a capsule sequentially generating the elements in the ratios of KFE determined.

This involves arranging the neutrons to form a backbone in a fractal pattern consistent with the fractal mathematics, arranging a proton core in a fractal pattern outside of the neutron backbone to balance consistent, and arranging the electron shell in a fractal pattern associated with the protons.

1. The process of fusion is initiated by generating neutrons from the neutron generator above the target location, generating a proton core around the neutrons and an electron shell around the protons, and compressing the resulting virtual capsule.
2. Organization and Compression is achieved by generating compressive situations by a combination of fractal magnetic effect and laser beams according fractal compression arrangement and steps.
3. igniting foam around the capsule to compress the proton-electron shell is also possible designing it to push information towards the interior location where the reaction is to occur but in the fractal steps and design suggested.
4. The fusion method for Hydrogen can be seen as having an inner collapse modeled on spiraling down according to MI (5, 3, 2, 1; possibly from multiple elements; and from two sides to get pairing; the next layer (protons and electrons) collapsing according to fpix (7, 5, 3, 1); possibly changing the direction of compression of the MI/Fpix/2\hat{ }n compression at fractal stepped stages to get to the Neutron collapse; and according to 2\hat{ }n compression overall. One example is compression with foam to generate plasma to shape with the pathways defined by the foam using changing thickness, where the ignition of the foam is focused to create KFE features. This same is done electromagnetically, mechanically, or explosively (as with laser or magnetic compression where using new models of magnetism allows for compression and decompression to be better designed).

In explosives one target is to focus on the “breathing” nature of explosives involved with this changing valuation (1, −3, 5, −7) where you can see expansion according to 2\hat{ }n but with changing compressive direction which can be targeted to increase or decrease effects by plugging in fractal elements dimensionally positioned around the explosive to increase or decrease the effects from that position. Since all fractals transitions occur in the same way, this can be used in chemistry, electronics, etc.

For fusion compression, dealing simultaneously with fpix and MI compression can be done by having the MI collapse-shaped capsule within the fpix-shaped capsule. Fractal modeling includes a move from a flat, 2-d capsule towards a 3-d capsule for neutrons by lasers pushing reactants in two dimensions and then adding the third dimension as they compress.

This mimics the idea of curved or circular flow which allows the orbital flow of atomic and subatomic ct-sates to collect information or disperse information along the fractally relevant dimensional line focused by a fractally shaped atomic or preatomic wall.

The neutron fractal alignment matches the arrangement fractally suggested of overlapping spirals (1:2:1, then 2:2 at right angles, then 3:3 at right angles). A spherical proton core can be compressed around this neutron arrangement. Since individual neutrons and protons cannot be controlled, these may be held as groups in ratios so that there are a bundle of neutrons surrounded by a bundle of protons even though the matrix of each type will be impure and the same is true of having the electrons exterior to this and the overall arrangement can be maintained as plasma to provide stability and separation until it is balanced to form atoms.

The process is to shape, virtually and otherwise, the reaction chamber to sequentially push highly compressed hydrogen into a more compressed state to provide a neutron backbone with proton stabilization in conjunction with fields to get the protons balanced with electrons.

Magnets can be used to reduce or add space. An outer inverter shell could contain this arrangement, requiring the entire arrangement be within a shell of overlapping magnetic fields, seen at large scale at intersecting magnetosphere/solar plasma between the earth and the sun, to isolate magnetic effects.

Shaping and concentration at scales where we can work can be used to position states along a path of reaction defined by, for example, iron filings having locations, widths, or separations within the magnetic fields consistent with the small scale fractally relevant transitions to encourage the KFE combinations.

This may be defined in terms of a process.

A method for controlling atomic and molecular stability, for fusion and chemistry comprising the steps of:
a. identifying the geometrical shift locations from the group comprised of fpix-based to Fibonacci-based and different base numbering-based geometry,
b. identifying the 2\hat{ }n transitions and matrices holding information states with a matrix and sub-matrix of information states;
c. manipulating the information states by changing the pre-time change and component information states, and the 2\hat{ }n cages and proximity of matrices to control the stability of the larger information states including neutrons, atoms, and molecules; controlling plasma concentrations in the form of information exchange between neutron, proton, proton positron and electron matrices make or dissolve bonds targeting the different dimensional features and information state exchanges and pre-time change within the different information states and matrices.

### Fission:

Utilizing KFE's ability to manipulate dimensional variations, control the release of pretime informational change, and design optimal matrices for energy generation, capture, transition and shielding.

General Energy: KFE energy converters can be used for staged fuels with fpix, MI, and exponential changes such as 1:1; 1:2:1:3:1:5 for burn times, heat, pressures, mixtures, and reactant matrices beginning and ending for any reaction to target specific fractal results.

Fractal design of matrices to take off energy in any form can extend the life of components by treating high energy particles as having high pretime change features which can be treated as frozen at quantum levels so that they may be diverted at these stages defined by fractal transitions to make them useful or less destructive.

### Chemistry:

This involves modeling chemical reactions along with categorization, interpretation and design using the algorithms that define atomic structure and information exchange between matrices.

The method for improving the chemistry can be applied, for example, to silicon or rare earths, by identifying the electromagnetic middle and geometric exchange of information states along fractal lines within the atoms and between the atoms and the matrix including the matrix of pre-time state changes “energizing” the atoms particularly where the matrices are within defined 2\hat{ }n compression matrices. The process includes the following steps:

a. identifying the fractal structure of the chemicals and then manipulating different fractal combinations including designing the desired matrices of the original and end matrices desired.
b. The fpix-controlled structures of the matrices within the MI matrices for all states below the neutron and the transition states to MI geometry at neutron bonding targets the fusion of information states from proton level compression to neutron compression and between neutrons which can include changing one or more of the identified key fractal elements in terms of the dimensional characteristics.

A method for controlling neutron stability, comprising the steps of:

a. identifying the geometrical shift from pi-based to MI based geometry at neutron bonding; identifying the 2\hat{ }n cages of compressive oriented materials that hold protons and electrons together as neutrons; and
b. manipulating the 2\hat{ }n cages to control the stability of the neutrons and protons; and
c. controlling geometric exchange interactions along fractal pathways defined by KFE to improve information exchange at various compression levels.

A method for controlling neutron stability, comprising the steps of:

a. Fractal features based on fpix and MI geometry at the atomic level change of geometric layout and compression and resulting base-numbered transitions.
b. By way of example, compression and decompression in total terms move from staggered areas of 2, 4, 8, 16, based on n=1, 2, 3, 4 in the equation 2\hat{ }n; but there are 2, 4, 8, and 16 intermediary stages with different base numbering.
c. Likewise, the buildup of concentrations within these compression states is along MI lines based on 2 times 1, 2, 3, 5 and in lower compression states according to 2 times 1, −3, 5, −7 in scale with the amount of force associated with these changes, including those seen as energy, based on the amount of information exchanged and the overall pretime change and the incremental steps.
d. Staged excitement by applying energy to the materials in a staged manner using different wavelengths of light, different temperatures, or different pressures is an example, but when these are designed around key fractal elements, the size and efficiency, and location of these changes can be better targeted including understanding the type and amount of pretime change or force associated with the associated information exchange so that the amounts can be aligned to make them more efficiently exchanged and in quantities that get the most efficient exchanges.
e. This involves targeting different base numbering systems present for different geometries in the design process. Since compression is a two-way street, modeling can also involve inverting the transition of ct state features, ½, ¼, ⅛ or ½, ⅓, 1/−3, etc. can be used to change the direction of fpix or MI transitions.
f. Dimensional changes of ct1 (non-dimensional. linear string) to ct2 (two-dimensional accordion-like compression) and stepped transitions to the recognized 3-dimensional structures and curvature can be targeted.

A method for Fusion includes generating a virtual capsule for (laser inertial) fusion, comprising generating a neutron generator offset from a target location, bringing the neutrons to a target location within protons aligned based on fractal atomic modeling. Further, the neutrons are offset from the target location fractally related to the arrangement of the neutrons and protons shown in fractal modeling of key fractal elements for atoms. The neutron generator may be radioactive elements which may be held in a fractally relevant separation or something like an MTS-LANSCE-LANL neutron generator in the same way.

### Chemistry: Hydrogen Generation

Energy is a disentanglement inherent in decompression/unwinding at the level of compression where the features of energy (electromagnetic information exchanges) occur including as components of larger atoms that can be dispersed most effectively along lines and within areas defined by KFE.

The expansion of water, for energy generation or in pistons or for propulsion can be viewed in terms of changing the bonds between the oxygen and the hydrogen at different energies interpreted as different amounts of pretime change, particularly ct4t11 states, and the resulting changes in the electron orbitals. Electron sharing is viewed in terms of overlap as well as shared ct4t12 states as well as their associated ct4t11 composite magnetic effects (M+ to distinguish them from e−, the electron).

Fractal modeling removes the focus from charge to CT state sharing and the disruption of CT states to target separation of hydrogen bonds (remote) and proton sharing (close). This also means creating an environment conducive to O—O sharing and H—H sharing while also disrupting the alternative bonds as a single step or as sequential steps.

H20 separation layouts are modeled with 0.75 of radius moves from outer to middle, 1.25 of radius moves from inner to 1 rs inside. This shows AuT or normal tesla valves with the length and angles defined by lines or curves defined by fpix or mi spirals or the length being defined by the 2\hat{ }n exchange areas. The layout of (left) anodes and cathodes and (right) a cathode broken out in the same fashion. The process can be reversed at various levels defined by the iterated equations given which give rise to the KFE which includes timing to encourage H2 formation from the dissolved water.

The two features are exponential change, balance, 2f(n), especially based on fpix for the hydrogens and bonds, but moderated by the MI for the Oxygen neutron backbone.

The shaping of the electrodes, e.g., round to match 2\hat{ }n, hexagonal to match fpix or MI transitions at different stages can be used. There are multiple places to disconnect so designing for each, one is the hydrogen bonds which are remotely shared between oxygen, a balance with a comparable “AuT-positron” equivalent coming off the Oxygen atom; and the second is that sharing more directly with oxygen. This process can be effective at the surface of a catalyst. Starting with an initial separation through the introduction of one or more types of information followed by a change to a second frequency or concentration of information.

Overall, the hydrogen separation techniques can incorporate KFE for separation whether in membranes, steam reforming steps, or within atomic or molecular structures designed as catalysts to lower the energy necessary for high-purity hydrogen. The use of fractal features in the design of the electrodes can help to improve the efficiency of the separation process. Fractals are self-similar patterns that repeat at different scales, and this property can be used to create electrodes with a high surface area-to-volume ratio. This increased surface area will allow for more efficient contact between the electrodes and the water molecules, which will lead to a more complete separation of the hydrogen and oxygen.

This can be described with process steps. A method for defining and manipulating chemicals, comprising the following steps:

a. Breaking down any reaction into KFE, maximizing efficiencies based on KFE. Dimensional features can shift between the energy storage and energy return phases of a KFE based fuel cell, for example, maximizing these by taking advantage of the shifting geometries and modeling based on these shifting geometries involves additional process steps:
a. identifying the fractal structure of the chemicals characterized by key fractal elements including antenna lengths of the various bonds between atoms including solvents and carriers; doing the same for the mechanical containers and separators so that you have KFE information states of the surrounding matrix and the underlying base number systems inherent in the transition between KFE information states of atomic and molecular matrix.
b. defining the shared information viewed as overlap of neutrons MI spirals, resulting proton core structures, and resulting electron structures including applicable spacing between protons and electrons and lower information states during the compressive and decompressive changes.
Model of a simple carbon-oxygen, hydrogen-oxygen reaction showing how packet size can be addressed based on the fractal sizes of the released information. You must take the shared information of two oxygen atoms into account in measuring the net information change.
c. Manipulating the fractal structure of the chemicals by changing fractal features of the atoms from the group comprised of, the initial overlap of neutrons, fractally relevant and defined areas of information exchange for the atoms and sub-atomic particles of the reaction including those with pre-time change seen as energy, the net and component fractal compression for an area of the matrix defined as the net compression and decompression for information states during a specific length of quantum change, base numbering for the different KFE and dimensional changes resulting for the matrix and any sub-matrix in the reaction or the environment or solution within which the manipulation (reaction, solution, information exchange) occurs.

### Chemistry PFAS

Breaking down chemicals, as with PFAS, using staggered excitement, fpix, 2\hat{ }n, 2\hat{ }1/n, and MI staging breaks down and passes through filters where the areas are staggered in the same ways such as 2, 4, 8, 16 in scale or energy size or doping levels for catalysts to break down the bonds and includes surface shaping to accomplish the result as by spraying against the surfaces staggered in that same fashion. An excited fluorine bond is hit with an electromagnetic burst to break and to entrap free fluorine or further break an atom. Additionally, to break by 2\hat{ }n or fpix with mi bursts. Catching the resulting ions with the anode or cathode attraction is possible, timing is tied to the pre-time fractally staged excitation of de-excitation.

Staged excitement is a technique that can be used to manipulate the fractal structure of materials by applying energy to the materials in a staged manner, done by using different pretime states with different amounts of wavelengths of light. but pre-time changes in magnetism also can be used with wavelike properties when viewed from the standpoint of quantum time, different temperatures, or different pressures when viewed from the standpoint of changing fractal structures within atoms with pre-time fixed features only appear as heat when viewed from a time perspective. To break down PFAS, the method manipulates the fractal structure of PFAS to destabilize the 2\hat{ }n cages holding protons and electrons together as neutrons. This would encourage the PFAS to break down into smaller, less harmful molecules.

The central first circle overlapping the ⅔ of each first Fibonacci arm forms a locking mechanism holding the f-series spirals together and allowing for the next stage of compression. Impact to 2\hat{ }n circles take out bonding “energy” ct states causing a powdering effect in metals which can be mimicked at atomic scales.

### Electronics:

This involves designing electronic reactions as being driven by the concept of energy as the amount of pretime change in a system as expressed at the level of the photon and concentrated in the form of electrons as a part of fpix based transitions interacting with protons, also as fpix based transitions and neutrons as MI bonded full compression states.

Being pretime, we only see the effects of the “magnetic” portion of the energy solution. Electrons are witnessed as t13 states T13, which are made up of t12 states as electrons. Circulating electrons are the magnetic ct4t11 STATES witnessed as rotated at 90 degrees and half size reflecting their exponential information content (approximately 1/10th that of an election) and their offset orientation which leads to the elliptical appearance of the cloud of information.

The pretime change inherent in all states are viewed as net effects. Our ability to get post-time “work” from pretime “change” is energy, an effect. The more pre-time changes within a given distance, the shorter the wavelength; and the more energy.

### Quantum Computing:

1) Time is stop-frame animation reflecting movements of dimensional features that are comprised of less compressed information than photons. “Compression of information” is a complicated concept, but mathematically defined in broad terms.
2) By defining time in terms of dimensional change, you can eliminate some of the “probability” aspects of QC by replacing probability with pre-time change, ultimately getting to quantum change but since that is possible, we get closer to quantum change and use these approximations to make the results using probability more accurate.

Using dimensional change in place of post time dimensional change for interpreting (vs drawing) qubits; even if averaging is necessary, can be used to fix the data.

Quantum computing replaces probability with estimated pretime change and changes in electrons based on fractal elements. It is possible to create a baseline or average baseline for pretime change in the qubit or qubit matrix by heating or adding pretime elements (e.g., energy/photons) to the qubit (think electron) or qubit matrix and/or cooling, allowing a drop in the pretime elements.

### Classification of Key Fractal Elements in Groups

Fractal balance, defined as an alignment of at least two higher compression CT states about at least one AuT fulcrum comprised of lower compression CT states by way of sharing the fulcrum.

AuT fulcrums are defined as lower compression CT states at the sharing of information location between at least two higher CT states.

The categorization of AuT matrix with key fractal elements includes 1) CT state content, 2) amount of CT states within the content; 3) relative dimensional size to at least one-second matrix, 3) locational area from the perspective of the time of generated by pretime change of the at least one matrix; 4) 5) fulcrum locations; and the amount of pretime change of the CT states, compression and decompression tendency within a matrix,

Absorption and spewing of CT states allows treating all dimensional transitions as force. Collision and reaction also reflect the exchange of information. Force can be categorized as the result of net compression/decompression (winding or unwinding) of CT states as viewed from post time CT state perspectives.

Fuse length is an example of memorized effects being carried forward, the fuse of any fpix solution being based on fpix absolute value and a total of states headed either towards or away from positive or negative can combine with others similarly situated to give either compressive effects if they interact, or decompressive if the solutions push them apart. The combination of solutions leading to folding which associates in two dimensions information can be seen as the initial step combining information to create compression states. This is the first state creating Dimension and Curvature defined by the number of compressed solutions of fpix for a matrix giving the 3-D matrix impression within the galaxy. At larger compression scale, one technique is to use the direction and fulcrums of compression and to maximize or minimize CT state exchanges to transfer information and maximize work.

### AI: Security

AI is built on algorithms, the better the algorithms the better the AI. KFE algorithms better interpret and categorize complex data matrices, leading to more accurate predictions, improved machine learning models, and advanced problem-solving capabilities. The process involves determining the KFE changes expected from the process and measuring deviations at its most simple. Since deviations may continue, the extent of these, i.e., change over quantum change and then change over time can be measured and actions set up based on these.

Deviations in processing occur during cyber threats. At scale these affect fractal systems and logic, circumventing the logic of the SSAI. Fractal science can be used to scale deviations from fractal baselines to determine the extent to which threats appear allowing the evaluation of the reaction of SSAI to different deviations and even to design systems to identify potentially harmful deviations which reflect the introduction or attempted introduction of a threat.

Trusted and secure validation and verification strategies to ensure that training data is not poisoned or inaccurate. All data is fundamentally fractal and since we have identified the mathematics behind these fractal transitions and fractal equivalence allows for approximation of results. The underlying fractal mathematics is uniquely suited to programming. N=−1*n is the fractal bit definition for changing information in computers corresponding to the slightly more complicated mathematics of fpix which is responsible for dimension building and ultimately curvature. These features can be combined to identify where bad data (not fractal equivalent, not interpreted according to the fractal modeling or inconsistent with fractal modeling) is being introduced, not necessarily to exclude it, but to weight it and bring awareness to uncategorizable data.

As the operations move away from fractal modeling, they will become less accurate.

### AI-Characterization

Fractal science can be used to scale data deviations from the normal line required by KFE changes to determine the extent to which data threats appear allowing the evaluation of the reaction of different deviations and even to design systems to identify potentially harmful deviations which reflect the introduction or attempted introduction of a threat to ensure that output and training data is not poisoned or inaccurate.

All data is fundamentally fractal and since we have identified the KFE mathematics behind these fractal transitions and fractal equivalence allows for approximation of results. The underlying fractal mathematics is uniquely suited to programming. N=−1*n is the fractal bit iterated equation for changing information in computers corresponding to the more complicated mathematics of fpix. These features can be used to identify where bad data (not fractal equivalent means bad) is being introduced, not to necessarily to exclude it, but to weight it and bring awareness to uncategorizable transitional data.

This process can be extended to continuous monitoring capabilities to secure development of models throughout the ML lifecycle; and to improve transparency and assurance of code, data, labels, and labeling processes. As programming driven operations move away from fractal modeling, they become less accurate. Identifying these changes is a critical step in this process.

### Big Data

A comprehensive method and system for organizing data can be based on fractal mathematics and dimensional features. By categorizing data within the framework of fractal science, including the application of fractal mathematics to space, time, forces, atoms, molecules, and larger structures, the system offers a robust approach to effectively organizing complex and unstructured data based on KFE and processing the data and data organization and use around KFE.

KFE can be used to build a fractal-based table of contents based on KFE based transitions, compression states and any other resulting KFE dimensional features. Data outliers (not showing fractal consistency according to KFE) are indicators of quality assurance issues. Fractal organization necessitates the determination of a common fractal link, enabling the breakdown of heterogeneous features into discernible fractal elements including suggested averages.

Comparing expected KFE determined outcomes to those seen can be used to provide accessibility, organization, and value of empirical data and organize or categorize it to improve prediction of electromagnetic, chemical and biological functions by reducing the parameters pf the data down to KFE and manipulating the data via programming based on the KFE elements alone and as reflected in the larger matrix of data under consideration.

“Unstructured metadata” is absent here due to the inherent categorization of all dimensional features within the universe under fractal mathematics. Time arises from dimensional changes resulting from pre-time dimensional states, facilitating the incorporation of unstructured data into the fractal model. Dynamic systems are classified based on the initial fractal matrix, the interaction of fractal elements, pretime changes, absorption, spew, and resulting fractal matrix.

The Neutron modeling of the PTE demonstrates using fractal formulas in graphical representations, specifically defining the structure of atoms, and reducing computational complexity while integrating mathematical models with observed structures and features. This can be shown with KFE representations of magnetism to show how the modeling can be used to visualize pre-time information changes in view of larger scale fractal equivalents.

Reconciling non-fractal science with fractal mathematics, the system emphasizes the evolution of both empirical models and fractal math for improved data organization and understanding.

Through a comprehensive mapping of equivalences between systems, including historical events and natural selection, within the framework of fractal mathematics, the proposed method and system offer a robust and efficient approach to data organization, paving the way for enhanced understanding and analysis in various fields of research and data management.

Empirical data related to atomic interactions for one or more of explosives, hydrogen and fuel cell chemistry, battery chemistry, advanced materials, semiconductor/silicon chemistry, rare earth chemistry, magnetic materials, PFAS chemistry, electronic elements, catalysts, matrices holding reactants, shielding/protecting/containment elements, elements tied to alignment of reactants, and elements tied to energy extraction can be categorized around KFE and associated patterns especially over quantum change bundles (up to and including time based, but including the pre-time changes) and developing fractal atomic structure models including describing transitions between energy and matter based on the KFE to predict behavior.

This system comprises a database storing empirical chemistry data related to atomic interactions; a software module configured to: access the empirical chemistry data; extract features from the data and categorize them based on KFE fractal features, identifying possible KFE transitions to create simulations and may also include comparing the simulations to empirical data; identifying discrepancies between empirical data and refining the fractal models to reconcile differences with empirical data.

### Military Tech

The modeling can be designed to increase absorption, dispersion, targeted reflection, based on information state transitions along KFE designed parameters. This involves the steps of creating and receiving information states according to key fractal elements of both the photonic level and larger KFE as well as those above those levels.

### Explosives and Shielding

The invention provides a method for using fractal modeling to increase or reduce the effectiveness of explosives. The method comprises the steps of: Developing a fractal model of the explosive matrix. Identifying key fractal elements in the explosive matrix. Modifying the key fractal elements to increase or disrupt the explosive fractal changes.

Explosives and even the expansion of water into snow crystals occur as 2\hat{ }n compression and decompression and for this reason focusing on those transitions allows for energy management.

The explosion itself results from 2\hat{ }n decompression of information, something unique to fractal modeling and the changes in the matrix attributable to dust or other contaminates can be viewed as irregularities in the otherwise regular 2\hat{ }n fractal exponential decompression matrix which is staged so that interference, dust for example, within the different exponential stages can be viewed as impacting the smooth absorption and spew of information associated with the fractal transition of regular and explosive reactions, including modifications based on specifics of the KFE of the interfering matrix. The explosions appear vibrational, with ⅓ transitions inward, for example, between 2\hat{ }n expansions, and/or concentrations at specific levels between 2\hat{ }n expansions ending or beginning at those locations. There are two parts of the matrix, the explosive reaction, and the environment in which the reaction takes place, both of which change during the quantum steps of the transformation. This includes the amount of pre-time change at various stages within the matrix.

A method for using fractal modeling to change the effectiveness of explosives, the method comprising the steps of:

a. Developing a fractal model of the explosive matrix based on the KFE and transitions in the KFE of the explosive and surrounding area; modifying the key fractal elements to increase or disrupt the explosive reaction at any stage both together and separately.

1. A method for analyzing and manipulating information states defined as parts dimensional features comprised of combined solutions of fpix to design electromechanical, physics and chemical interactions in structural components, and wherein combing solutions is compression and breaking down combined solutions is decompression of dimension comprising:
a. Identifying the KFE fractal information content of dimensional manifestations using a fractal-based model, on the observed bit fpix up to neutrons;
b. Identifying the KFE fractal information content of atomic dimensional manifestations using a fractal-based model, on the geometry of MI beginning at neutron bonding;
c. applying the KFE fractal information content to analyze and manipulate matrices of fractal states defined using fpix and dimensional transitions.

2. The method of claim 1 further comprising a method for enhancing energy conversion efficiency, comprising:
a. configuring elements control reactions and electromagnetic dimensional transitions between a pi-defined geometry and a MI-defined geometry at neutron bonding; wherein the fractally mandated dimensional transitions based on key fractal elements from the set comprising 2f(n), n=n+1, particularly to 3 places, fpix, Fibonacci (MI) or MI/fpix transitions, and wherein f(n) is from the group comprising fpix and MI, and 2\hat{ }n steps of intermediary compression steps and where force represents the exchange of information states.

3. The method of claim 1 wherein the dimensional manifestations are from the group comprising the process of designing fractal changes to enhance a process from the group consisted of AI algorithms, energy manipulation, material manipulation, energy storage, energy generation, machine learning, chemistry, predictive science, and quantum computing.

4. The method of claim 1, further comprising treating changes in photon and smaller divisions of dimensional representation as creating time as dimensional change based on changes in iterated equations within Key Fractal Elements and treating energy as these changes at the photon level.

5. The method of claim 4 further comprising;
a. identifying at least one first pretime information state as a qubit;
b. applying an operation to the qubit;
c. identifying at least one average change in the qubit;
d. doing programming based on the changes in the at least one qubit to clarify the probability measurements of qubit change used in quantum computing.

6. The method of claim 1 further comprising using iterated equation with data science to modify AI software to maximize the accessibility, organization, and value of empirical data and to allow this data to be used along with fractal modeling to predict force, chemical and biological functions, and further comprising the steps of:
a. using a fractal model to model energy, atomics, chemical, and biological systems, categorizing data from the sub-atomic through the molecular systems,
b. Developing a classification system to cross-characterize existing, empirical non-fractal data within sub-atomic, atomic, chemical, and biological data with Key fractal elements,
c. Breaking down data into its key fractal elements,
d. Integrating the key fractal elements into AI software to allow the AI software to use the fractal model to access, organize, and analyze empirical data,
e. Using algorithms giving rise to Key fractal elements to design, categorize, organize, or predict force, chemical and biological outcomes.

7. The method of claim 6, further comprises tracking information in the form of compressible fractal states, comprising: a. Identifying the compression states within a data set. b. Utilizing key fractal elements within the identified compression states. c. Tracking the fused compression states for purposes of data management in computing functions.

8. The method of claim 7, wherein the computing functions include sorting, organizing, and tracking data in search engines according to the key fractal elements within the data.

9. The method according to claim 1, related to time treated as a quantum dimensional change in fractal states within the matrix, modifying at least one fractal within the matrix to change the transitions between the fractal element compression to obtain lower and higher compression states from the group comprising increasing compression, decreasing compression, destabilizing compression or stabilizing compression.

10. The method of claim 2, further comprising:
a. changing reaction geometries from fpix based geometries to MI based geometries to encourage fusion.

11. The method of claim 10, further comprising the step of treating neutron bonding as based on neutron fractal elements being balanced on either side by lower compression states acting as fulcrums to encourage the fusion process and wherein the neutrons are centrally located on the fulcrum relative to protons.

12. The method of claim 11, further comprises structuring the neutron and proton concentrations separated based on two dimensional fpix geometry for protons and electrons and three-dimensional MI geometry for the neutrons of the fulcrum.

13. The method of claim 12, further comprising structuring sequential compression of the fused elements according to 2\hat{ }n changes in the sequence.

14. The method of claim 13, wherein the process includes a concentration of the fusion elements according to the order set forth utilizing equipment to sequentially generate the desired compression and decompression of key fractal elements to obtain fusion.

15. The method of claim 1, wherein collections of dimensional states comprise matrices and there is a rate of fractal change within each matrix and wherein the process further comprises changing the rate of lower compression state changes according to key fractal elements within the matrix.

16. The method of claim 15, wherein the process of manipulating fractal changes further includes changing the ratio of changing fractal states to non-changing fractal states at different points within the matrix for changing the rate of lower compression state exchanges between higher compression states to create changes like the resulting matrix.

17. The method according to claim 1, wherein the key fractal elements are utilized in changing the ratio of fractal states to non-changing fractal states at different points within the matrix for changing the rate of lower compression state exchanges between higher compression states to create changes in the resulting matrix; and wherein the process of manipulating fractal changes further includes targeting fractal changes to enhance a process from the group consisting of energy manipulation, material manipulation, energy storage, energy generation, machine learning, chemistry, predictive science, and quantum computing and wherein the compression states at which energy becomes apparent are treated as a transition between pre-time fractal compression states and post-time fractal compression states, and wherein changing comprises treating time as change in the pre-time fractal compression states viewed from the post time fractal compression states.

18. A method for developing an AI framework of AI neurons, comprising the steps of:
a) Formulating AI neuron models based on fractal mathematics, incorporating self-repeating patterns and scalable properties;
b) Designing said AI neurons to exhibit hierarchical organization and relationships based on KFE of different elements of the framework;
c) Implementing said AI neurons in a fractal AI framework, enabling efficient resource allocation and accelerated convergence;
d) Training and validating the fractal AI framework using standard AI datasets to assess performance against conventional AI models, and;
c) Applying the fractal AI framework to diverse AI tasks, including image recognition, natural language processing, and reinforcement learning, to achieve unprecedented levels of performance and versatility.

19. A computational system comprising an AI framework of AI neurons, wherein said AI neurons are designed using a combination of information physics and fractal mathematics to exhibit self-similarity and scalability properties, comprises:
a. AI framework being interconnected hierarchically to create versatile neural network architectures capable of dynamic learning and continuous adaptation, designing said AI neurons to use key fractal elements in the expression of hierarchical connectivity and self-adaptation, and training and validating the fractal AI framework using key fractal elements for hierarchy.

20. The method of claim 1, further comprises using key fractal elements, which affect change in CT states,
a. interprets, matrices of CT states to optimize polymerization reactions, enables efficient hydrogen release, and storage capacity, to improve hydrogen production methods, to optimize hydrogen extraction reactions, to design radiation matrices for fusion energy production, to improve fission processes, to optimize chemical reactions enabling enhanced reaction kinetics, to enable increased battery capacity for improved charging and discharging rates, and; to enhance qubit stability, and increase computational power to optimize AI algorithms.
